(19) United States
(12) Patent Application Publication
(10) Pub. No.: US 2022/0032272 A1
(43) Pub. Date: Feb. 3, 2022

(54) CATALYST FOR DEHYDRATION REACTION OF PRIMARY ALCOHOLS, METHOD FOR PREPARING THE SAME AND METHOD FOR PREPARING ALPHA-OLEFINS USING THE SAME

(71) Applicant: KOREA INSTITUTE OF ENERGY RESEARCH, Daejeon (KR)

(72) Inventors: Kee Young KOO, Daejeon (KR); Un-ho JUNG, Daejeon (KR); Young-eun KIM, Jeju-si (KR); HYO BEEN IM, Daejeon (KR); DONG-HYUN CHUN, Daejeon (KR); Min Hye YOUN, Daejeon (KR); Heon-do JEONG, Daejeon (KR); GEUN BAE RHIM, Daejeon (KR); Ji-chan PARK, Daejeon (KR); Dong-wook LEE, Daejeon (KR)

(21) Appl. No.: 17/386,555

(22) Filed: Jul. 28, 2021

(30) Foreign Application Priority Data
Jul. 31, 2020 (KR) 10-2020-0095651

(51) Int. Cl. B01J 23/02; B01J 21/04; B01J 37/02; B01J 37/08; C07C 1/24

(52) U.S. Cl. CPC B01J 23/02; B01J 21/04; B01J 37/0201; C07C 2521/04; B01J 37/088; C07C 1/24; C07C 2523/02; B01J 37/0236

(57) ABSTRACT

Provided are a catalyst for dehydration reaction of a primary alcohol, a method for preparing the same, and a method for preparing alpha-olefins using the same. According to the present invention, there is provided a catalyst for dehydration reaction of primary alcohols capable of adjusting the strength and distribution of Lewis acid sites (LASs) on a surface of an alumina catalyst to realize high selectivity to alpha-olefins as well as a high conversion rate in the dehydration reaction of primary alcohols. Therefore, high-purity alpha-olefins having a low isomeric yield fraction as well as a high conversion rate can be produced from the primary alcohols.

### CROSS-REFERENCE TO RELATED APPLICATIONS

This application claims priority under 35 U.S.C. § 119 to Korean Patent Application No. 10-2020-0095651, filed on Jul. 31, 2020, in the Korean Intellectual Property Office, the disclosure of which is incorporated herein by reference in its entirety.

### TECHNICAL FIELD

The following disclosure relates to a catalyst for a dehydration reaction of a primary alcohol, a method for preparing the same and a method for preparing an alpha-olefin using the same.

### BACKGROUND

Linear alpha-olefins (LAOs) are basic materials that are used in various chemical industries, such as copolymers of polyolefins, lubricants, plasticizing agents, and the like, and thus are effectively used in various chemical products. LAOs with a wide carbon range (C4 to C20) are mainly produced on a small scale on the basis of naphtha crackers, or produced by oligomerization of ethylene. In recent years, a demand for LAOs has been increasing steadily. In particular, LAOs having carbon atoms of even numbers (such as C₆, C₈, C₁₀, and the like) may be widely applied to co-monomers of linear low-density polyethylene (LLDPE), copolymers of polyolefin elastomers (POEs), lubricating base oils, and the like. However, because a commercialization process is very limited, the production of high-purity LAOs is one of techniques that are very important in the chemical industry.

1-octene was commercialized from Sasol based on a process for tetramerization of ethylene. Specifically, Sasol produces 1-octanol through a hydroformylation reaction and a hydrogenation reaction of 1-heptene extracted from a Fisher-Tropsch stream, and subjects the 1-octanol to a dehydration reaction to produce 1-octene. However, such a process produces a large amount of by-products such as dioctyl ether (DOE), isomers of 1-octene, and the like. Separation of DOE from 1-octene is relatively easily achieved because a difference of the boiling points thereof is greater than or equal to 100° C., but cis- or trans-octene that is an isomer of 1-octene has a small boiling point difference of approximately 1 to 2° C., and its separation process is complicated due to the other similar physical properties. Therefore, there is still a demand for research on a dehydration reaction of 1-octanol having high selectivity to 1-octene and a low production yield of isomers.

Non-Patent Document 1 reports that Lewis acid sites on a surface of an Al₂O₃ catalyst are highly associated with the conversion of 1-octanol during a dehydration reaction. Also, Non-Patent Documents 2 to 4 report that Al₂O₃ and SiO—Al₂O₃ catalysts have similar effects on the dehydration reaction of isopropanol and 1-butanol. However, such a highly acidic catalyst produces an excessive amount of isomers in the dehydration reaction of alcohol as previously described above. In this regard, Non-Patent Document 5 reports that this is because the Lewis acid sites that are active sites of the catalyst re-adhere to the produced alpha-olefins for an isomerization reaction.

Based on this background, the present inventors have deepened research on the assumption that the key point of the high LAO selectivity in the dehydration reaction of alcohol is to control the Lewis acid sites of the catalyst.

### RELATED ART DOCUMENTS

### Non-Patent Documents

Non-Patent Document 1: Fuel, Volume 256, 15 Nov. 2019, 115957
Non-Patent Document 2: Applied Catalysis, Volume 26, 1986, Pages 295-304
Non-Patent Document 3: Catalysis Today, Volume 5, Issue 2, April 1989, Pages 121-137
Non-Patent Document 4: Applied Catalysis, Volume 70, Issue 1, 1991, Pages 307-323
Non-Patent Document 5: Applied Catalysis, Volume 31, Issue 2, 15 Jun. 1987, Pages 361-383

### SUMMARY

An embodiment of the present invention is directed to providing a catalyst for a dehydration reaction of primary alcohols, and a use thereof.

Specifically, there is provided a catalyst for a dehydration reaction of a primary alcohol capable of adjusting the strength and distribution of Lewis acid sites (LASs) on a surface of an alumina catalyst to realize high selectivity to alpha-olefins as well as a high conversion rate in the dehydration reaction of primary alcohols

Specifically, an embodiment of the present invention is directed to providing a method for preparing the catalyst for a dehydration reaction of a primary alcohol as described above.

Specifically, another embodiment of the present invention is directed to providing a method for preparing an alpha-olefin from a primary alcohol using the catalyst for a dehydration reaction of a primary alcohol as described above.

In a general aspect, there is provided a catalyst for a dehydration reaction of a primary alcohol, wherein barium oxide (BaO) is supported on an alumina carrier according to the present invention. In this case, the catalyst may be a catalyst in which 0.1 to 1.5% by weight of barium oxide is supported based on the total weight of the carrier.

The catalyst according to one embodiment of the present invention may convert a primary alcohol into an alpha-olefin.

The catalyst according to one embodiment of the present invention may convert a primary linear alcohol having 4 to 20 carbon atoms into an alpha-olefin.

The catalyst according to one embodiment of the present invention may convert 1-octanol into 1-octene.

In the catalyst according to one embodiment of the present invention, the alumina carrier may be a gamma-alumina carrier, a delta-alumina carrier, a theta-alumina carrier, an eta-alumina carrier, or an alpha-alumina carrier.

In another general aspect, there is provided a method for preparing the catalyst for a dehydration reaction of a primary alcohol as described above. Specifically, the method for preparing a catalyst for a dehydration reaction of a primary alcohol includes mixing a barium precursor with an alumina carrier to impregnate the alumina carrier; and drying and calcining the resulting mixture.

In the method for preparing a catalyst for a dehydration reaction of a primary alcohol according to one embodiment of the present invention, the barium precursor may be one or a mixture of two or more selected from barium nitrate, barium nitrite, barium acetate, barium sulfate, barium carbonate, and the like.

In still another general aspect, there is provided a method for preparing an alpha-olefin from a primary alcohol, which includes subjecting a primary alcohol to a dehydration reaction while continuously adding the primary alcohol in the presence of the catalyst for a dehydration reaction of a primary alcohol as described above.

In the method for preparing an alpha-olefin from a primary alcohol according to one embodiment of the present invention, the primary alcohol may be supplied in an evaporated form.

In the method for preparing an alpha-olefin from a primary alcohol according to one embodiment of the present invention, the subjecting of the primary alcohol may be performed at 250 to 500° C.

In the method for preparing an alpha-olefin from a primary alcohol according to one embodiment of the present invention, the subjecting of the primary alcohol may be performed by supplying the primary alcohol at a liquid hourly space velocity (LHSV) of 4 to 60 h⁻¹ at 250 to 500° C.

In the method for preparing an alpha-olefin from a primary alcohol according to one embodiment of the present invention, the subjecting of the primary alcohol may be performed by supplying the primary alcohol at a liquid hourly space velocity of 4 to 60 h⁻¹ at 250 to 500° C. In this case, the conversion rate of the primary alcohol is greater than or equal to 60%, and the selectivity of the alpha-olefin may satisfy a range of 50% or more.

In addition, in the method for preparing an alpha-olefin from a primary alcohol according to one embodiment of the present invention, when the dehydration reaction is performed under the conditions as described above, the yield of the alpha-olefin is greater than or equal to 40%, and the purity of the alpha-olefin may satisfy a range of 50% or more.

### BRIEF DESCRIPTION OF THE DRAWINGS

FIG. 1 is a schematic diagram of a dehydration reaction of 1-octanol according to the present invention.

FIG. 2 is a graph showing the results of Examples and Comparative Examples according to the present invention, that is, a graph confirming an effect of a reaction temperature on the conversion rate of 1-octanol (LHSV=7 h⁻¹, and reaction temperature=300° C., 350° C., and 400° C.).

FIG. 3 is a graph showing the results of Examples and Comparative Examples according to the present invention, that is, a graph confirming an effect of a reaction temperature on the selectivity of 1-octene (LHSV=7 h⁻¹, and reaction temperature=300° C., 350° C., and 400° C.).

FIG. 4 is a graph showing the results of Examples and Comparative Examples according to the present invention, that is, a graph confirming an effect of a reaction temperature on the yield of 1-octene (LHSV=7 h⁻¹, and reaction temperature=300° C., 350° C., and 400° C.)

FIG. 5 is a graph showing the results of Examples and Comparative Examples according to the present invention, that is, a graph confirming an effect of a reaction temperature on the purity of 1-octene (LHSV=7 h⁻¹, and reaction temperature=300° C., 350° C., and 400° C.).

FIG. 6 is a graph showing the results of Examples and Comparative Examples according to the present invention, that is, a graph confirming an effect of a liquid hourly space velocity on the conversion rate of 1-octanol (reaction temperature=400° C.).

FIG. 7 is a graph showing the results of Examples and Comparative Examples according to the present invention, that is, a graph confirming an effect of a liquid hourly space velocity on the selectivity of 1-octene (reaction temperature=400° C.).

FIG. 8 is a graph showing the results of Examples and Comparative Examples according to the present invention, that is, a graph confirming an effect of a liquid hourly space velocity on the yield of 1-octene (reaction temperature=400° C.).

FIG. 9 is a graph showing the results of Examples and Comparative Examples according to the present invention, that is, a graph confirming an effect of a liquid hourly space velocity on the purity of 1-octene (reaction temperature=400° C.).

FIG. 10 is a graph showing the results of Examples and Comparative Examples according to the present invention, that is, a graph confirming an effect of a liquid hourly space velocity on the yield of dioctyl ether (DOE) (reaction temperature=400° C.).

FIG. 11 shows the py.-FTIR data of Examples and Comparative Examples according to the present invention.

### DETAILED DESCRIPTION OF EMBODIMENTS

Hereinafter, a catalyst for a dehydration reaction of a primary alcohol according to the present invention, a method for preparing the same, and a method for preparing an alpha-olefin using the same will be described in detail with reference to the accompanying drawings.

The drawings presented in this specification are shown as one example to sufficiently provide the scope of the present invention to those skilled in the art. Therefore, it should be understood that the present invention may be embodied in various forms, but is not intended to be limited to the drawings presented hereinbelow. In this case, the drawings may be shown in an exaggerated manner to make the scope of the present invention more clearly apparent.

Unless otherwise defined, the technical and scientific terms used in this specification have the same meaning as commonly understood by one of ordinary skill in the art to which the present invention pertains. In the following description and the accompanying drawings, a description of known functions and configurations, which may unnecessarily obscure the subject matter of the present invention, will be omitted.

Also, the singular forms “a,” “an,” and “the” used in the specification of the present invention and the appended claims are intended to refer to those including plural referents unless the context clearly dictates otherwise.

In addition, the units used without any particular comments in the specification of the present invention are based on weight. For example, the units of % or percentage refer to a percent (%) by weight or weight percentage.

Additionally, unless otherwise defined in the specification of the present invention, an average particle size of particles refers to D₅₀ obtained using particle size analyzer.

Also, a numerical range used in the specification of the present invention is meant to include its upper and lower limits and all possible combinations of all values falling within these limits, increments logically derived from the shapes and widths of defined ranges, all defined values thereof, and upper and lower limits of the numerical ranges defined in different types. For example, it should be interpreted that, when a content of a composition is in a range of 10% to 80%, specifically in a limited range of 20% to 50%, a numerical range of 10% to 50% or 50% to 80% is also described in the specification of the present invention. Unless otherwise particularly defined in this specification of the present invention, all values falling out of this numerical range that may occur due to the rounding off of the experimental errors or values also fall within the defined numerical ranges.

In addition, in the specification of the present invention, the expression “comprise(s)” is intended to encompass open-ended transitional phrases having an equivalent meaning with “contain(s),” “include(s),” “have,” “has,” and “is(are) characterized by,” and does not exclude elements, materials, or steps, all of which are not further recited herein. Also, the expression “consist(s) essentially of” means that one element, material, or step, which is not recited in combination with the other elements, materials, or steps, may be present at an amount having no unacceptably significant influence on at least one basic and novel technical idea of the invention. Also, the expression “consist(s) of” means the presence of only the elements, materials or steps defined hereafter.

Further, in the specification of the present invention, the term “conversion” refers to a process of producing an alpha-olefin through a dehydration reaction of a primary alcohol.

As stated in the “Background” section of the present invention, the present inventors have deepened research on the assumption that the key point of the high LAO selectivity in dehydration reaction of alcohol is to control Lewis acid sites of a catalyst. While deepening the research, the present inventors have found an effect of barium that is an alkali earth metal in adjusting the strength and distribution of Lewis acid sites (LASs) on a surface of a highly acidic alumina catalyst.

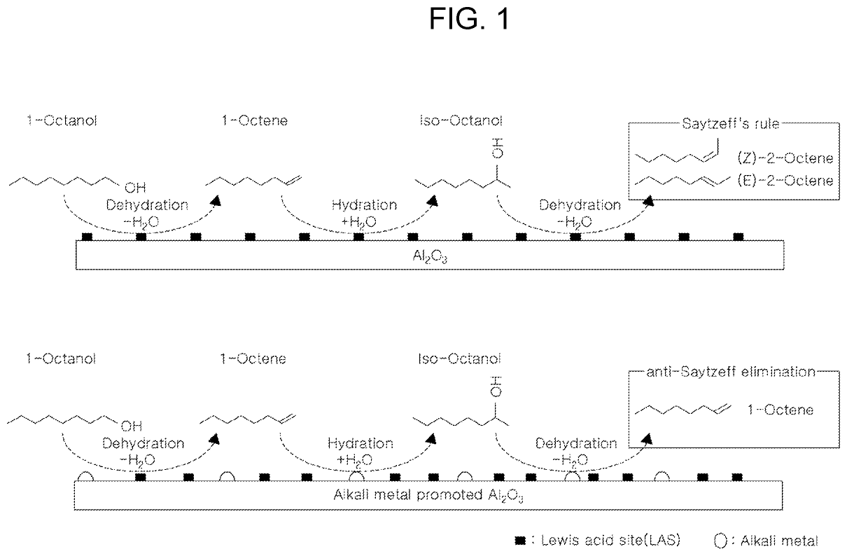

Specifically, the present inventors have found that an alumina catalyst in which barium oxide is supported at 1.5% by weight or less based on the total weight of the catalyst may provide a synergistic effect in conversion of a primary alcohol into an alpha-olefin through an anti-Saytzeff elimination in a dehydration reaction of the primary alcohol, and thus try to suggest the present invention (see FIG. 1).

Hereinafter, the present invention will be described in detail.

The present invention provides a catalyst for a dehydration reaction of a primary alcohol, wherein barium oxide is supported on an alumina carrier. In this case, the catalyst may be a catalyst in which 0.1 to 1.5% by weight of barium oxide is supported based on the total weight of the catalyst.

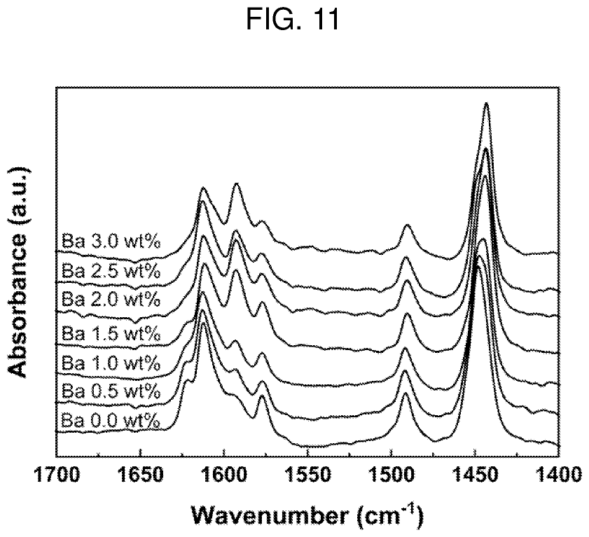

Referring to FIG. 11, it can be seen that a loading amount of barium oxide has an influence on Lewis acid sites of a highly acidic alumina catalyst. Specifically, pyridine in the alumina catalyst binds to strong, medium, and weak LASs at 1,621, 1,614, and 1,594 cm⁻¹, respectively.

Specifically, the catalyst for a dehydration reaction of a primary alcohol according to one embodiment of the present invention decreases the strong LAS strength (at 1,621 cm−1) and increases the weak LAS strength (at 1,594 cm−1) since the above-described loading amount of barium oxide is satisfied. Accordingly, the catalyst for a dehydration reaction of a primary alcohol according to the present invention is expected to realize a high conversion rate and selectivity of the alpha-olefin. On the other hand, it is confirmed that, when an amount of barium oxide exceeds the above-described loading amount of barium oxide, the strong LAS strength (at 1,621 cm−1) is not observed, and a significant decrease in the conversion rate is caused due to the production of an excessive amount of DOE, which is not desirable.

In the catalyst for a dehydration reaction of a primary alcohol according to one embodiment of the present invention, it is desirable that the loading amount of barium oxide is not limited as long as it satisfies a range of 0.1% by weight to 1.5% by weight, and may be widely varied and used according to the desired type of primary alcohol, the process conditions, and the like.

As one example, the loading amount of barium oxide may be greater than or equal to 0.3% by weight.

As one example, the loading amount of barium oxide may be greater than or equal to 0.51 by weight.

As one example, the loading amount of barium oxide is desirably in a range of 0.5 to 1.5% by weight, and most desirably satisfies a loading amount of 1.0 to 1.5% by weight.

In the catalyst for a dehydration reaction of a primary alcohol according to one embodiment of the present invention, the primary alcohol may be used with limitation as long as it is a primary linear alcohol having 4 to 20 carbon atoms. One nol-limiting example of the primary alcohol includes hexanol, heptanol, octanol, nonanol, decanol, undecanol, undecenol, dodecanol, tridecanol, tetradecanol, pentadecanol, hexadecanol, heptadecanol, octadecanol, and the like.

Preferably, the primary alcohol may be 1-octanol in the present invention. Therefore, the catalyst for a dehydration reaction of a primary alcohol according to the present invention may be specifically a heterogeneous catalyst for conversion of 1-octanol into 1-octene.

Also, in the catalyst for a dehydration reaction of a primary alcohol according to one embodiment of the present invention, the alumina carrier may be porous alumina. Also, the alumina carrier may be crystalline alumina or amorphous alumina. Desirably, the alumina carrier may be a gamma-alumina carrier, a delta-alumina carrier, a theta-alumina carrier, an eta-alumina carrier, or an alpha-alumina carrier. More desirably, the alumina carrier may be a gamma-alumina carrier.

As one example, the alumina carrier may have an average particle size in a range of 0.1 to 5.0 mm, specifically in a range of 0.5 to 3.0 mm, and more specifically in a range of 1.0 to 2.0 mm.

As one example, the alumina carrier may have a surface area of 500 m²/g or less. Specifically, the alumina carrier may have a surface area in a range of 50 to 400 m²/g, and more specifically in a range of 100 to 300 m²/g.

As one example, the alumina carrier may have a total pore volume of 1.0 cm³/g or less, specifically 0.9 cm³/g or less, more specifically 0.1 to 0.6 cm³/g, and most specifically in a range of 0.45 to 0.5 cm³/g.

As one example, the catalyst for a dehydration reaction of a primary alcohol may be a catalyst in which 0.1 to 1.5% by weight of barium oxide is loaded on the gamma-alumina carrier based on the total weight of the catalyst.

As one example, when the above-described loading amount of barium oxide is satisfied, the catalyst for a dehydration reaction of a primary alcohol does not cause a change in basic physical properties of the alumina carrier. Specifically, the basic physical properties may include a surface area of the alumina carrier, a total pore volume, a peak of whole detached CO₂, and the like.

According to the present invention, there is also provided a method for preparing the catalyst for a dehydration reaction of a primary alcohol as described above.

The method for preparing a catalyst for a dehydration reaction of a primary alcohol according to one embodiment of the present invention may include mixing a barium precursor with an alumina carrier to impregnate the alumina carrier; and drying and calcining the resulting mixture. Specifically, the impregnation may be performed through an incipient wetness method.

In the incipient wetness method, the barium precursor may be dissolved in a solvent in consideration of the loading amount of barium oxide, and the alumina carrier may be impregnated into the resulting mixture. In this case, the solvent is not particularly limited, and may be used as long as it is a solvent that may dissolve the barium precursor.

As one example, the solvent may be water, an alcoholic solvent, or a combination thereof. Also, a usage amount of the solvent should not exceed an amount that may be absorbed by the alumina carrier. In particular, the usage amount of the solvent is preferably the maximum amount that may be absorbed by the alumina carrier.

Next, the drying and calcining of the resulting mixture may be performed.

The drying method is not particularly limited, and may, for example, include heating the mixture at 90 to 150° C. for 5 hours to 24 hours to remove the solvent.

Also, the calcining method is not particularly limited, and may, for example, include calcining the mixture in a sealed heating space such as an oven. The calcining may, for example, be performed in an air atmosphere, but the present invention is not limited thereto.

As one example, the calcining may be performed at a temperature of 300 to 700° C. When this temperature range is satisfied, the barium oxide may be distributed in the alumina carrier with a proper particle size of barium oxide, which is favorable to the activity of the catalyst. Also, the calcining is desirably performed for an hour to 24 hours.

As one example, the alumina carrier may be used after being pre-calcined at 300 to 1000° C. in the air.

In the method for preparing a catalyst for a dehydration reaction of a primary alcohol according to one embodiment of the present invention, the barium precursor may include one or a mixture of two or more selected from barium nitrate, barium nitrite, barium acetate, barium sulfate, barium carbonate, and the like. In this case, it is desirable that a usage amount of the barium precursor may be varied and used with various conditions compositions as long as it falls within a range that does not fall out of the desired loading amount of barium oxide in the present invention.

Also, the present invention provides a method for preparing an alpha-olefin from a primary alcohol using the above-described catalyst for a dehydration reaction of a primary alcohol. To selectively prepare an alpha-olefin in high yield, it is very important to select a reaction temperature and a supply velocity of a reactant as well as a composition of the above-described catalyst for a dehydration reaction of a primary alcohol.

Specifically, the method for preparing an alpha-olefin from a primary alcohol according to one embodiment of the present invention may include subjecting a primary alcohol to a dehydration reaction while continuously adding the primary alcohol in the presence of a catalyst for a dehydration reaction of a primary alcohol in which 0.1 to 1.5% by weight of barium oxide is supported on the alumina carrier, based on the total weight of the catalyst. Here, the catalyst for a dehydration reaction of a primary alcohol may be present in a state in which the catalyst is filled into a fixed-bed reactor, and the shape, length, and the like of the fixed-bed reactor may widely vary depending on a purpose thereof. Also, the catalyst for a dehydration reaction of a primary alcohol may also be applied to forms which are introduced so that the catalyst is present in a reactor for a conventional dehydration reaction.

The primary alcohol may be supplied to the catalyst present in the fixed-bed reactor in an evaporated form, and a dehydration reaction may be performed while continuously adding the primary alcohol. In this case, this step may be performed under the condition of a temperature of 250 to 500° C., and the dehydration reaction of the primary alcohol is performed accordingly.

As one example, the dehydration reaction may be specifically performed at 280 to 450° C., and more specifically at 300 to 400° C.

As one example, the dehydration reaction may be performed at the above-described temperature for 1 to 5 hours.

To minimize a wide variation in range of the reaction temperature according to the latent heat, the primary alcohol may be supplied in an evaporated form at this temperature. Also, when the primary alcohol is supplied to the catalyst in an evaporated form, the primary alcohol may be supplied with a carrier gas. The carrier gas may, for example, be a nitrogen gas.

As one example, the carrier gas may be supplied at a velocity of 20 to 80 mL/min per 0.5 mL of the catalyst for a dehydration reaction of a primary alcohol. In this case, the velocity of the carrier gas may be determined in proportion to the volume of the catalyst for a dehydration reaction of a primary alcohol, but the present invention is not limited thereto.

In the method for preparing an alpha-olefin from a primary alcohol according to one embodiment of the present invention, the dehydration reaction may be performed by supplying the primary alcohol at 250 to 500° C. and a liquid hourly space velocity of 4 to 60 h⁻¹. Also, the supply of the primary alcohol may mean that the primary alcohol is supplied to the catalyst in an evaporated form.

As one example, in the dehydration reaction, the liquid hourly space velocity may be specifically in a range of 7 to 56 h⁻¹, more specifically in a range of 7 to 35 h⁻¹, and most specifically in a range of 14 to 28 h⁻¹.

As one example, the dehydration reaction may be performed through a column filled with the catalyst for a dehydration reaction of a primary alcohol. In this case, the dehydration reaction may be performed by supplying the primary alcohol in an evaporated form.

As one example, the dehydration reaction may be performed by allowing the primary alcohol to pass through a column containing a filer layer including the catalyst for a dehydration reaction of a primary alcohol at the liquid hourly space velocity and in the temperature range as described above. The specific operation conditions of the dehydration reaction in the column may be changed into the conditions known in the art, and then may be used without limitation in consideration of the reaction temperature and the supply velocity of the reactant as described above. Also, the product obtained through the dehydration reaction may be transferred to a distillation apparatus equipped with a cooling machine and a sorting machine, and may be sorted and recovered using a difference in boiling point. Thereafter, the recovered liquid-phase or gas-phase product may be quantified by GC-FID.

In the method for preparing an alpha-olefin from a primary alcohol according to one embodiment of the present invention, the conversion rate tends to decrease at a low reaction temperature (300° C.) with an increasing loading amount of barium oxide in the dehydration reaction. In particular, when the loading amount of barium oxide is greater than 2.0% by weight, the conversion rate of the alpha-olefin greatly decreases to less than 40%. Also, the conversion rate tends to be improved with an increasing reaction temperature in the dehydration reaction, and the alpha-olefin seems to have significant selectivity and purity as compared to pure alumina.

As one example, a 1.5% by weight Ba/Al₂O₃ catalyst has a 1-octene selectivity of 63.9% and a 1-octene purity of 64.1% at 400° C. at which most of the primary alcohol is converted. On the other hand, the pure alumina has a 1-octene selectivity of 34.4% and a 1-octene purity of 34.5%.

In the method for preparing an alpha-olefin from a primary alcohol according to one embodiment of the present invention, the conversion rate tends to decrease with an increasing liquid hourly space velocity in the dehydration reaction. In particular, when the loading amount of barium oxide is greater than 2.0% by weight, this tendency is more prominent. On the other hand, the purity and yield of the alpha-olefin tend to be improved with an increasing liquid hourly space velocity in the dehydration reaction. Such results are significant compared to the pure alumina.

As one example, when the dehydration reaction is performed by supplying the primary alcohol at 300 to 400° C. and a liquid hourly space velocity of 7 to 56 h⁻¹, the conversion rate of the primary alcohol is greater than or equal to 60%, and the selectivity of the alpha-olefin may satisfy a range of 50% or more. In addition, according to the present invention, the above-described conversion rate and selectivity of the alpha-olefin are satisfied, and the yield of the alpha-olefin is greater than or equal to 40%, and the purity of the alpha-olefin may also satisfy a range of 50% or more at the same time.

Hereinafter, the present invention will be described in detail with reference to embodiments thereof. It should be understood that the embodiments are merely intended to describe the present invention in more detail, but are not intended to limit the scope of the present invention.

### Preparative Example 1

### Preparation of 0.5% by Weight of Ba/Al₂O₃ Catalyst

Spherical Al₂O₃ particles (d=1 mm; Sasol) contained elemental impurities such as 0.020% Si, 0.015% Fe, 0.015% Ti, and 0.002%. Na. After the particles were pre-calcined at 500° C. for 6 hours in the air, barium was loaded by an incipient wet impregnation method, using barium nitrite (99%, Sigma-Aldrich) as a barium precursor, so that an amount of the barium was 0.5% by weight based on the total weight of the catalyst. Then, the resulting mixture was heated at 110° C. for 12 hours to remove water, and then calcined at 500° C. for 4 hours in the air to prepare a 0.5% by weight Ba/Al₂O₃ catalyst.

Also, the physical properties of the prepared catalyst were determined. The results are listed in Table 1 below.

### Preparative Examples 2 to 5

1.0% by weight to 3.0% by weight Ba/Al₂O₃ catalysts were prepared in the same manner as in Preparative Example 1.

Also, the physical properties of the prepared catalysts were determined. The results are listed in Table 1 below.

TABLE 1

| | Items | | | | | | |
|---|---|---|---|---|---|---|---|
| | | Preparative | Preparative | Preparative | Preparative | Preparative | Preparative |
| | γ-Al₂O₃ | Example 1 | Example 2 | Example 3 | Example 4 | Example 5 | Example 6 |
| BaO | 0 | 0.5 | 1.0 | 1.5 | 2.0 | 2.5 | 3.0 |
| loading | | | | | | | |
| amount (% | | | | | | | |
| by weight) | | | | | | | |
| Ba | 0.00 | 0.50 | 0.97 | 1.36 | 1.85 | 2.38 | 2.89 |
| content (% | | | | | | | |
| by weight)^{a} | | | | | | | |
| Surface | 158 | 161 | 161 | 160 | 160 | 144 | 138 |
| area (m²/g)^{b} | | | | | | | |
| Total pore | 0.47 | 0.48 | 0.47 | 0.47 | 0.47 | 0.44 | 0.43 |
| volume | | | | | | | |
| (cm³/g)^{b} | | | | | | | |
| Whole | 0.036 | 0.039 | 0.040 | 0.049 | 0.061 | 0.067 | 0.076 |
| detached | | | | | | | |
| CO₂ | | | | | | | |
| (mmol/g)^{c} | | | | | | | |

^{a}Measured by ICP-MS (ELAN DRC II, Perlan-Elmer).
^{b}Estimated from N₂ adsorption at −196° C. in BET (Brunauer-Emmett-Teller) and BJH (Barrett-Joyner-Halenda) methods using BELSORP-max (BEL Japan, Inc.).
^{c}Estimated from CO₂-TPD peaks using a BELCAT-B (BEL Japan, Inc.) system.

A BET surface area, a total pore volume, and a pore size distribution of a Ba/Al₂O₃ catalyst having a Ba content as an initial amount as measured by ICP-MS described in Table 1 were measured by a BET-BJH analysis. As a result, the specific surface area and the total pore volume of the 2.0% by weight Ba/Al₂O₃ catalyst were also maintained. However, the BET surface areas of the 2.5% by weight Ba/Al₂O₃ and 3.0% by weight Ba/Al₂O₃ catalysts decreased due to the clogging of pores by Ba.

Also, the crystal structures of the Ba/Al₂O₃ catalysts having various Ba contents were observed. As a result, a significant change in XRD peaks was not observed. Based on such results, it was expected that the peaks characteristic of the crystal structures were not observed due to the low crystallinity of barium oxide.

### Comparative Example 1

Gamma-alumina (γ-Al₂O₃) thermally treated under an air atmosphere of 550° C. was used as the catalyst for a dehydration reaction of a primary alcohol.

A dehydration reaction was performed using a fixed-bed reactor. A quartz tube (diameter: 9.5 mm and length: 522 mm) reactor was used, and the total length of the catalyst layer was 51 mm. For the sufficient evaporation and uniform distribution of 1-octanol, SiC (2 g, 1 mm, Goodfellow (NP-KX-201, NS)) was quantitatively supplied, pre-heated at 300° C., and then injected into the catalyst layer.

The activity of the catalyst was evaluated at an LHSV of 7 to 56 h⁻¹ and 300′C to 400° C. for 3 hours under an atmospheric pressure. Also, the product was quantified by GC-FID (Hewlett-Packard 5890 series gas chromatograph, 60 m/0.25 mm HP-5 capillary column).

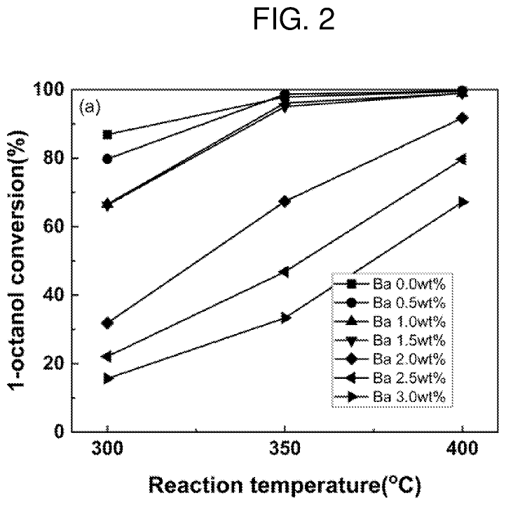

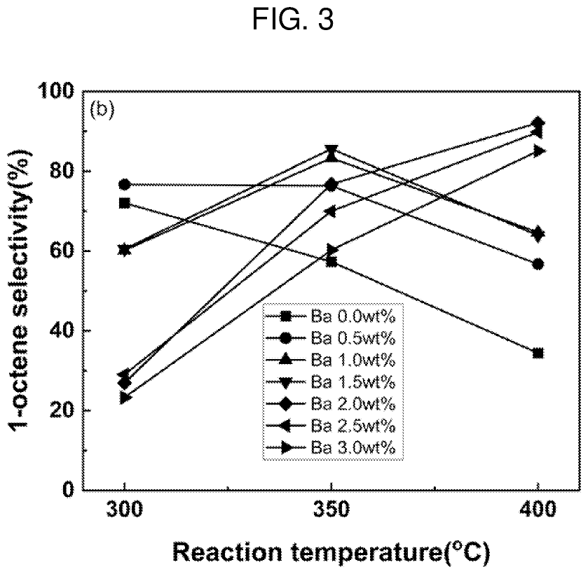

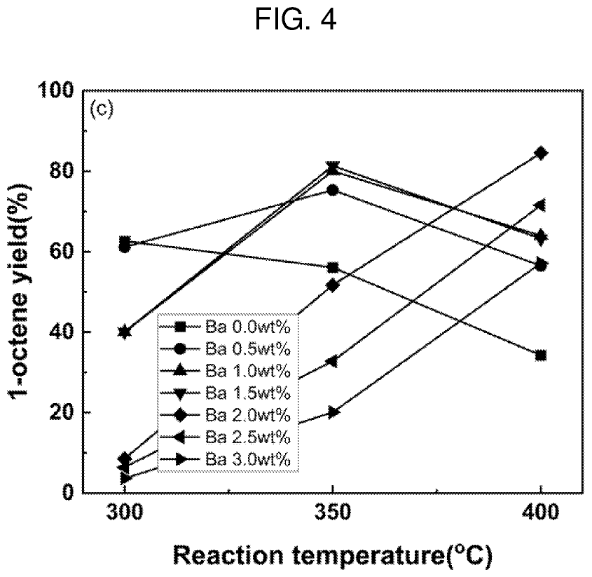

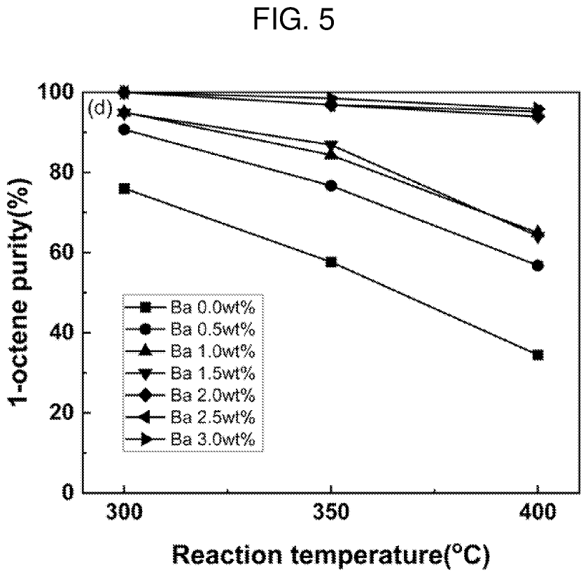

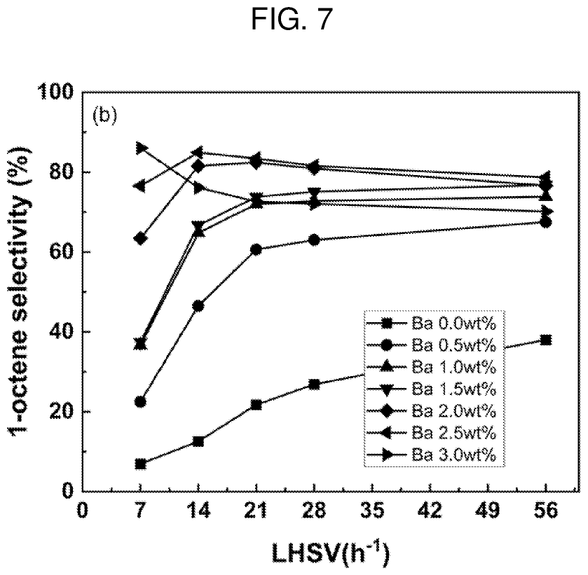

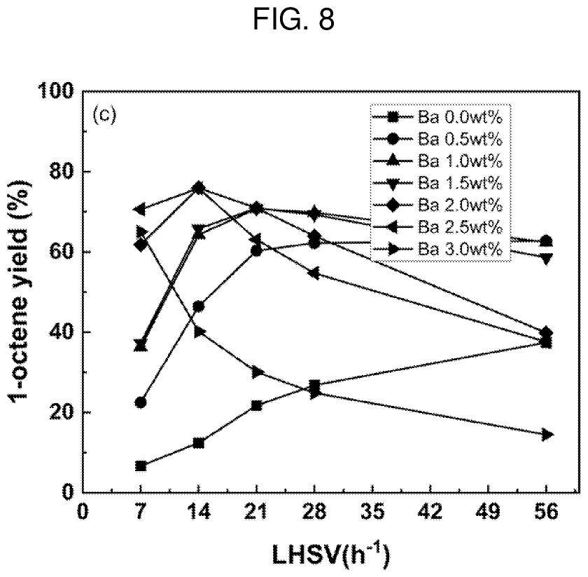

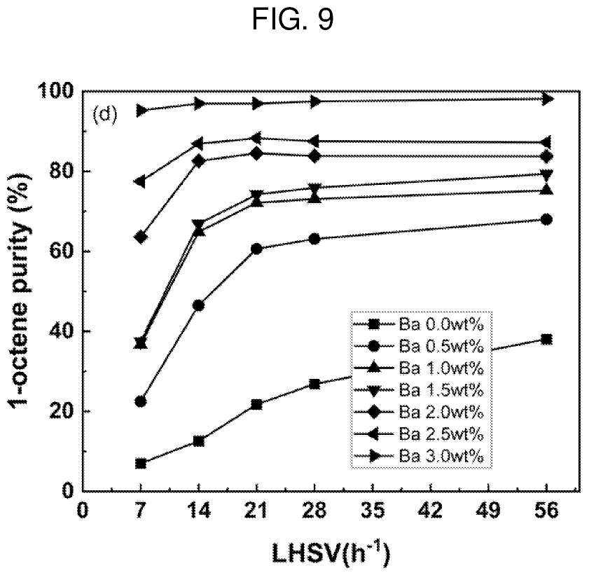

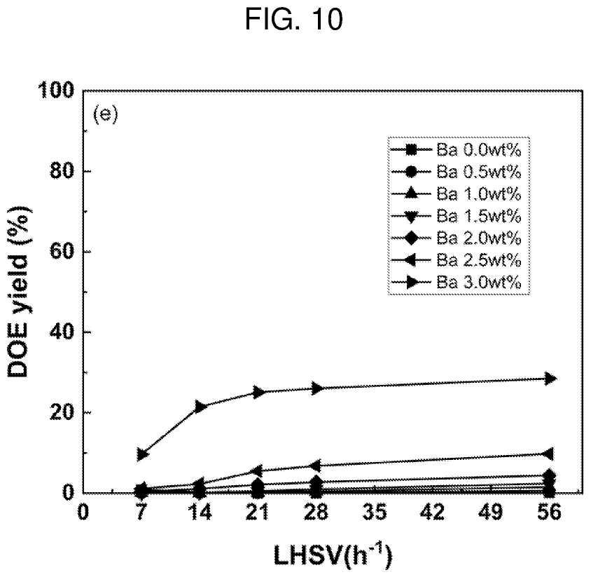

The following Equations are used to calculate the conversion rate of 1-octanol, the selectivity of 1-octene, the yield of 1-octene, the purity of 1-octene, and the selectivity of DEO. The results obtained from such calculations are shown in FIGS. 2 to 10 below.

$$\begin{matrix}Conversion rate ( \% ) of 1 \text{-} octanol = \frac{Number of moles of converted 1 \text{-} octanol}{Initial number of moles of 1 \text{-} octanol} \times 100 & [ Equation 1 ] \\ Selectivity ( \% ) of 1 \text{-} octene = \frac{\begin{matrix}Number of moles of 1 \text{-} octanol \\ converted into 1 \text{-} octene\end{matrix}}{Number of moles of converted 1 \text{-} octanol} \times 100 & [ Equation 2 ] \\ Yield ( \% ) of 1 \text{-} octene = \frac{\begin{matrix}Number of moles of 1 \text{-} octanol \\ converted into 1 \text{-} octene\end{matrix}}{Initial number of moles of 1 \text{-} octanol} \times 100 & [ Equation 3 ] \\ Purity ( \% ) of 1 \text{-} octene = \frac{\begin{matrix}Number of moles of 1 \text{-} octanol \\ converted into 1 \text{-} octene\end{matrix}}{\begin{matrix}Number of moles of 1 \text{-} octanol \\ converted into octene\end{matrix} } \times 100 & [ Equation 4 ] \\ Selectivity ( \% ) of DEO = \frac{\begin{matrix}Number of moles of 1 \text{-} octanol \\ converted into dioctylether ( DOE )\end{matrix}}{Number of moles of converted 1 \text{-} octanol} \times 100 & [ Equation 5 ]\end{matrix}$$

### Examples 1 to 3 and Comparative Examples 2 to 4

Dehydration reactions were performed in the same manner as in Comparative Example 1 using the catalysts for dehydration reaction of a primary alcohol as listed in Table 2 below.

The activities of the catalysts were evaluated at an LHSV of 7 to 56 h⁻¹ and 300° C. to 400° C. for 3 hours under an atmospheric pressure. The results are shown in FIGS. 2 to 10 below.

TABLE 2

| | Items | | | | | | |
|---|---|---|---|---|---|---|---|
| | Comparative | | | | Comparative | Comparative | Comparative |
| | Example 1 | Example 1 | Example 2 | Example 3 | Example 2 | Example 3 | Example 4 |
| | Catalyst | | | | | | |
| | | Preparative | Preparative | Preparative | Preparative | Preparative | Preparative |
| | γ-Al₂O₃ | Example 1 | Example 2 | Example 3 | Example 4 | Example 5 | Example 6 |
| BaO | 0 | 0.5 | 1.0 | 1.5 | 2.0 | 2.5 | 3.0 |
| loading | | | | | | | |
| amount (% | | | | | | | |
| of weight) | | | | | | | |

FIGS. 2 to 5 below show the activities of the Ba/Al₂O₃ catalyst having various Ba contents. Because the Ba content increased at a low reaction temperature (300° C.), the conversion rate of 1-octanol decreased. In particular, when the Ba content was greater than 2.0% by weight, the conversion rate of 1-octanol significantly decreased to less than 40%. Also, when the Ba content is less than or equal to 1.5% by weight, the strength of LASs decreased compared to the pure alumina, but the strong LASs were still exposed to a portion of a surface of the catalyst. On the other hand, when the Ba content was excessive, the strong LASs were hardly observed. That is, it can be seen that the Ba/Al₂O₃ catalysts were involved in the regulation of the strength and distribution of the strong LASs, indicating that the Ba/Al₂O₃ catalysts had an influence on the selectivity, yield, and purity of 1-octene, and the like as well as the conversion rate of 1-octanol.

Also, as the reaction temperature increased, the conversion rates of 1-octanol for all of the prepared catalysts were improved. Specifically, it was confirmed that the 0.5 to 1.5% by weight Ba/Al₂O₃ catalysts showed the similar conversion rates of 1-octanol at a temperature of 350° C. or higher, and the Ba/Al₂O₃ catalysts with the above-described Ba contents showed higher selectivity of 1-octene and purity of 1-octene, compared to the pure alumina. Also, the 1.5% by weight Ba/Al₂O₃ catalyst had a 1-octene selectivity of 63.9% and a 1-octene purity of 64.1% at 400 at which most 1-octanol was converted, and the pure alumina had a 1-octene selectivity of 34.4% and a 1-octene purity of 34.5%. It can be seen from such results that the production of octene isomers was inhibited by inhibition of reabsorption of 1-octene.

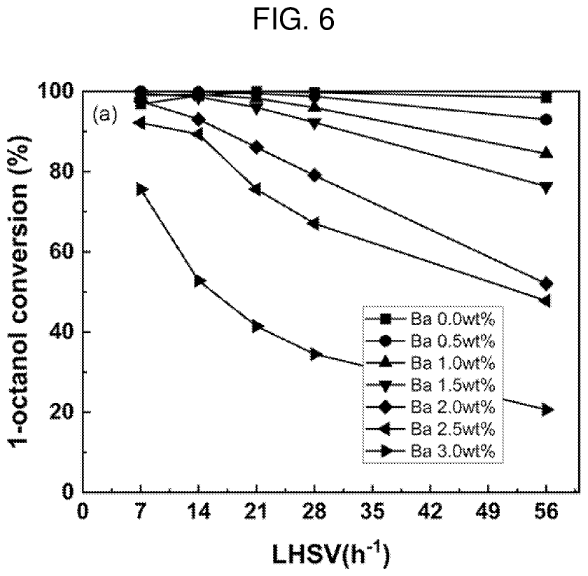

FIGS. 6 to 10 below show the activities of the catalysts according to the liquid hourly space velocity at a reaction temperature of 400° C. at which the conversion rate of 1-octanol is almost 100%. Unlike the pure alumina, the Ba/Al₂O₃ catalysts had a decreased conversion rate of 1-octanol as the liquid hourly space velocity increased to 21 h⁻¹ or more. In the case of the 2.0% by weight Ba/Al₂O₃ catalyst, the conversion rate of 1-octanol suddenly decreased with an increasing liquid hourly space velocity. Also, the selectivity of 1-octene and the purity of 1-octene increased in a similar manner with an increasing liquid hourly space velocity.

Meanwhile, the pure alumina has a large amount of the strong LASs on a surface thereof showed the lowest purity of 1-octene. The Ba/Al₂O₃ catalysts according to the present invention showed excellence in that the Ba/Al₂O₃ catalysts maintained excellent selectivity of 1-octene and purity of 1-octene even when the liquid hourly space velocity increased to 21 h⁻¹ or more. After some of the strong LASs were coated with Ba, the Ba/Al₂O₃ catalysts showed high purity of 1-octene without producing isomers in the dehydration reaction. Also, the selectivity of 1-octene to the 2.03 by weight Ba/Al₂O₃ catalyst decreased with an increasing liquid hourly space velocity due to the presence of DOE as the by-product rather than the formation of the isomers. In fact, it is anticipated that this was because the strong LASs and barium oxide acted as active sites for etherification of 1-octanol at the same time, and the production of DOE was improved when the Ba content was greater than or equal to 2.0% by weight.

It was confirmed from such results that the 1.5% by weight Ba/Al₂O₃ catalysts were the most suitable catalysts to produce 1-octene through a dehydration reaction of 1-octanol because the 1.5% by weight Ba/Al₂O₃ catalysts minimized the production of by-products and showed the highest purity of 1-octene as well.

The catalyst for a dehydration reaction of a primary alcohol according to the present invention can control Lewis acid sites of an alumina carrier and effectively prevent the reabsorption of produced alpha-olefins because 0.1 to 1.5% by weight of barium oxide is supported on the alumina carrier, based on the total weight of the catalyst. Thus, according to the present invention, the high conversion rate of the primary alcohol and the high selectivity to alpha-olefins can be realized. That is, the catalyst for a dehydration reaction of a primary alcohol according to the present invention in which barium oxide was supported at the above-described loading amount has a synergistic effect on the conversion rate of the primary alcohol and the selectivity of the alpha-olefins.

The conversion of the primary alcohol using the catalyst for a dehydration reaction of a primary alcohol according to the present invention can be performed with a conversion rate of up to 99.7%, and high-purity alpha-olefins having a low isomeric yield fraction can be obtained. Specifically, the yield fraction of the isomers can be at least 3.7%. Therefore, the yield fraction of the alpha-olefin can be shown to be up to 84.3%.

The conversion of the primary alcohol using the catalyst for a dehydration reaction of a primary alcohol according to the present invention shows high purity and selectivity to alpha-olefins even when the reaction temperature increases. Also, the catalyst for a dehydration reaction of a primary alcohol according to the present invention has an advantage in that higher-purity alpha-olefins can be obtained because the conversion rate can be rather reduced but a decrease in isomeric yield fraction of alpha-olefins is caused as the liquid hourly space velocity increases.

Although the effects are not explicitly mentioned in the present invention, the effects described in the specification, which are expected by the technical features of the present invention, and provisional effects thereof are handled as described above in the specification of the present invention.

What is claimed is:

1. A catalyst for a dehydration reaction of a primary alcohol, wherein 0.1 to 1.5% by weight of barium oxide is supported on an alumina carrier, based on the total weight of the catalyst.

2. The catalyst of claim 1, wherein the catalyst is used to convert the primary alcohol into an alpha-olefin.

3. The catalyst of claim 2, wherein the primary alcohol is a primary linear alcohol having 4 to 20 carbon atoms.

4. The catalyst of claim 3, wherein the primary alcohol is 1-octanol.

5. The catalyst of claim 1, wherein the alumina carrier is a gamma-alumina carrier, a delta-alumina carrier, a theta-alumina carrier, an eta-alumina carrier, or an alpha-alumina carrier.

6. A method for preparing the catalyst for a dehydration reaction of a primary alcohol defined in claim 1, comprising:
mixing a barium precursor with an alumina carrier to impregnate the alumina carrier; and
drying and calcining the resulting mixture.

7. The method of claim 6, wherein the barium precursor is selected from barium nitrate, barium nitrite, barium acetate, barium sulfate, and barium carbonate.

8. A method for preparing an alpha-olefin from a primary alcohol, comprising:
subjecting a primary alcohol to a dehydration reaction while continuously adding the primary alcohol in the presence of a catalyst for a dehydration reaction of a primary alcohol in which 0.1 to 1.5% by weight of barium oxide is supported, based on the total weight of the catalyst.

9. The method of claim 8, wherein the adding of the primary alcohol comprises supplying the primary alcohol into the catalyst in an evaporated form.

10. The method of claim 8, wherein the subjecting of the primary alcohol is performed at 250 to 500° C.

11. The method of claim 10, wherein the subjecting of the primary alcohol comprises supplying the primary alcohol at a liquid hourly space velocity of 4 to 70 h⁻¹.

12. The method of claim 11, wherein a conversion rate of the primary alcohol is greater than 60%, and
a selectivity of alpha-olefin is greater than 50%.

13. The method of claim 12, wherein a yield of the alpha-olefin is greater than 40%, and
a purity of the alpha-olefin is greater than 50%.
